(19) United States
(12) Patent Application Publication
(10) Pub. No.: US 2014/0066975 A1
(43) Pub. Date: Mar. 6, 2014

(54) Pad for Imaginary Acupuncture Therapy and a Method for Preparing the Same

(71) Applicant: Tae Woo Yoo, Chungcheongnam-do (KR)

(72) Inventor: Tae Woo Yoo, Chungcheongnam-do (KR)

(21) Appl. No.: 13/746,829

(22) Filed: Jan. 22, 2013

(30) Foreign Application Priority Data
Aug. 29, 2012 (KR) 2012-94620

(51) Int. Cl. A61B 17/34

(52) U.S. Cl. CPC A61B 17/34
USPC 606/204

(57) ABSTRACT

A pad for imaginary acupuncture therapy includes a cushion layer in an upper part, into which an acupuncture pin is inserted, a blocking plate for blocking the acupuncture pin from penetrating into the skin in a lower part of the pad and which is attached to spots on the body suitable for acupuncture, and a protection film on the cushion layer for confirming reuse of the pad, such that the protection film functions to prevent secondary infection caused by reusing the pad and to protect against contamination due to scattering of crumbled materials from the surface of the pad.

### BACKGROUND OF THE INVENTION

The present invention is related to a pad for imaginary acupuncture therapy and a method for preparing the same. Particularly, the present invention is related to a pad for an imaginary acupuncture therapy which comprises a cushion layer in an upper part, in which an acupuncture pin is inserted, a blocking plate for blocking the acupuncture pin from penetrating into the skin in a lower part and which is attached to spots on the body suitable for acupuncture, and a protection film on the cushion layer for confirming the reuse of the pad. The protection film functions to prevent secondary infection caused by reusing the pad and to protect from contamination due to scattering of crumbled materials from the surface of the pad.

Generally, acupuncture therapy performed in oriental medicine is to stimulate spots relevant to subject disease among spots distributed on the body suitable for acupuncture by a fine needle to control the energy of the body and thereby cure the subject diseases. Such acupuncture therapy has been recognized as a certified superior medical therapy from the old times.

In the aforementioned acupuncture therapy, needles are inserted into several spots and their lengths are different in order to cure even one disease. Also, the inserted depths of the needles are different depending on the disease to be cured. Due to these properties, a patient who is treated for the first time or has some hyper-response to acupuncture feels fear to acupuncture therapy. Thus, the acupuncture therapy was apt to be avoided.

An example of the recent alternative medical therapy is an imaginary therapy using the placebo effect in which a fake drug composing sugar or starch with no medicinal effect is administered to a patient as if a true drug to give an effect of improving the symptom of the patient.

Said imaginary therapy has proved, as research results, the production of chemicals which bring an improvement of a healing mechanism of the body when we feel good emotions such as pleasure, happiness, peace of mind and composedness, and reversely, the production of chemicals which brings our body into the disease and death when we feel bad emotions such as fear, anger, sense of guilt and impotent feeling. Thus, such imaginary therapy has been systematically applied to clinical tests of several symptoms to result in remarkable effects of disease treatment.

Meanwhile, said imaginary therapy has been applied to the acupuncture therapy. The present applicant has suggested in the design application for a stimulation pad (Application No. 2011-0007093) which provides a curing effect by acupuncture simulation without inserting an acupuncture pin into the skin. Said stimulation pad comprises a cushion layer in an upper part, in which an acupuncture pin is inserted into, a hard blocking plate for blocking penetration of the acupuncture pin to the skin in a lower part, of which the bottom is attached with the skin via an adhesive.

However, in said stimulation pad, as the cushion layer for inserting acupuncture is made by sponge or expanded resin, it has been often reused without any indication of acupuncture insertion. Due to the reuse, the surface of the cushion layer is crushed, and thereby, fine crumbled materials are produced during the insertion and removing of the acupuncture pin. Therefore, said stimulation pad has some problems of secondary infection caused by reusing the pad and contamination from scattering of fine crumbled materials from the surface of the pad.

### SUMMARY OF THE INVENTION

The present invention is designed to solve the aforesaid problems of a conventional stimulation pad. Thus, the object of the present invention is to provide a pad for imaginary acupuncture therapy which prevents reuse of the pad and allows acupuncture to be performed in a clean condition, and a method for preparing the same.

In order to accomplish said objective, the pad for imaginary acupuncture therapy according to the present invention comprises a cushion layer in an upper part, in which an acupuncture pin is inserted, a hard blocking plate for blocking the acupuncture pin from penetrating into the skin in a lower part, of which the bottom is attached with the skin via adhesives, and a protecting film on the cushion layer for confirming the reuse of the pad by checking a penetration hole formed on the surface of the surface of said cushion layer caused by acupuncture.

The other object of the present invention is to provide various shapes of separated pads by preparing a basic pad for imaginary acupuncture therapy by laminating in order of a coating paper in a suitable size, a blocking plate with the same size as the coating paper where an adhesive is applied to the bottom thereof, a cushion layer with the depth sufficient for inserting an acupuncture pin, and a protection film in a shape of thin membrane, and then cutting the blocking plate, the cushion layer and the protecting film in an integrated state excluding the coating paper from the basic pad by using cutters having various pad shapes.

### BRIEF DESCRIPTION OF THE DRAWINGS

FIGS. 1a and 1b are a perspective view showing a separated process for preparing the pad for imaginary acupuncture therapy and a perspective view of the combined pad for imaginary acupuncture therapy according to the present invention, respectively.

FIGS. 2a and 2b are a perspective view showing the completed state of the pad for imaginary acupuncture therapy and a perspective view showing parts drawn out from the basic pad according to the present invention, respectively.

FIG. 3 is a cross section view showing the completed state of said pad for imaginary acupuncture therapy.

FIGS. 4a and 4b are a perspective view and a cross-section view showing the before and the after states of inserting an acupuncture pin into the pad for imaginary acupuncture therapy according to the present invention, respectively.

FIG. 5 is an illustrative view showing a using state of the pad for imaginary acupuncture therapy according to the present invention.

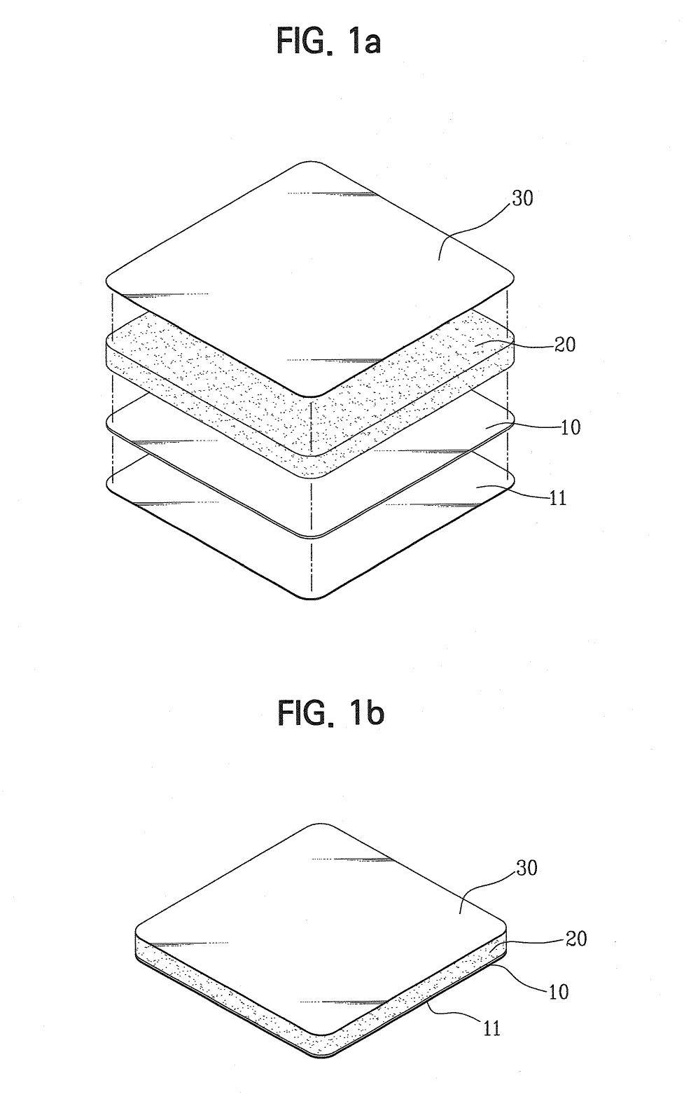

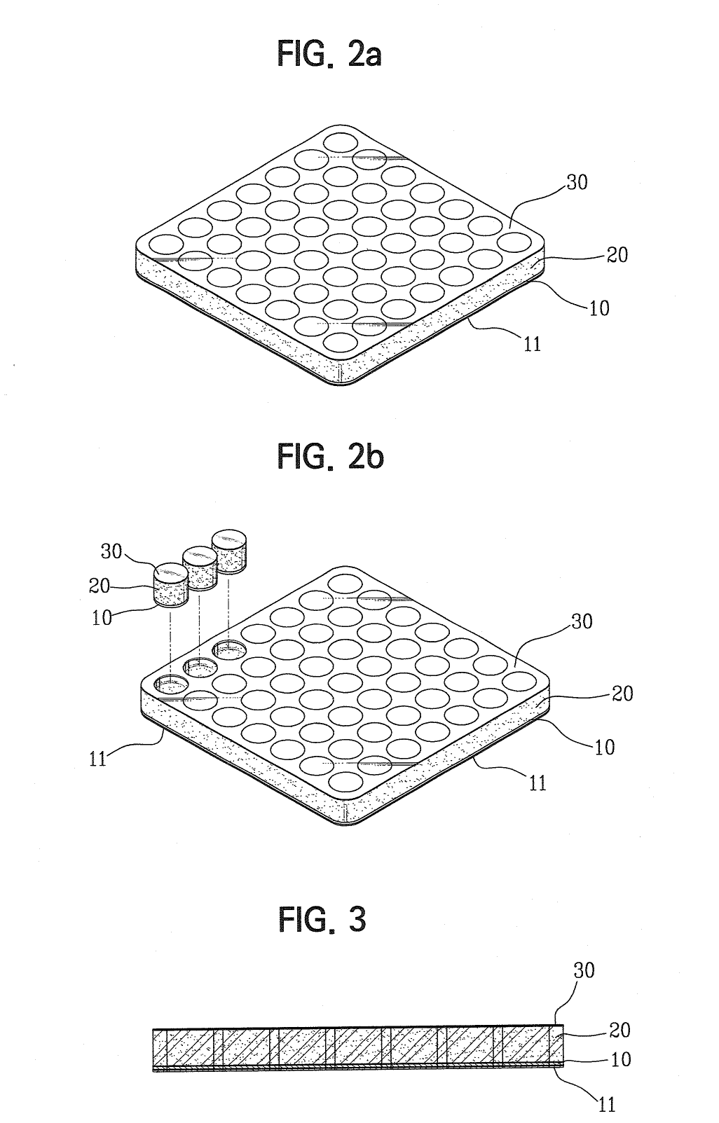

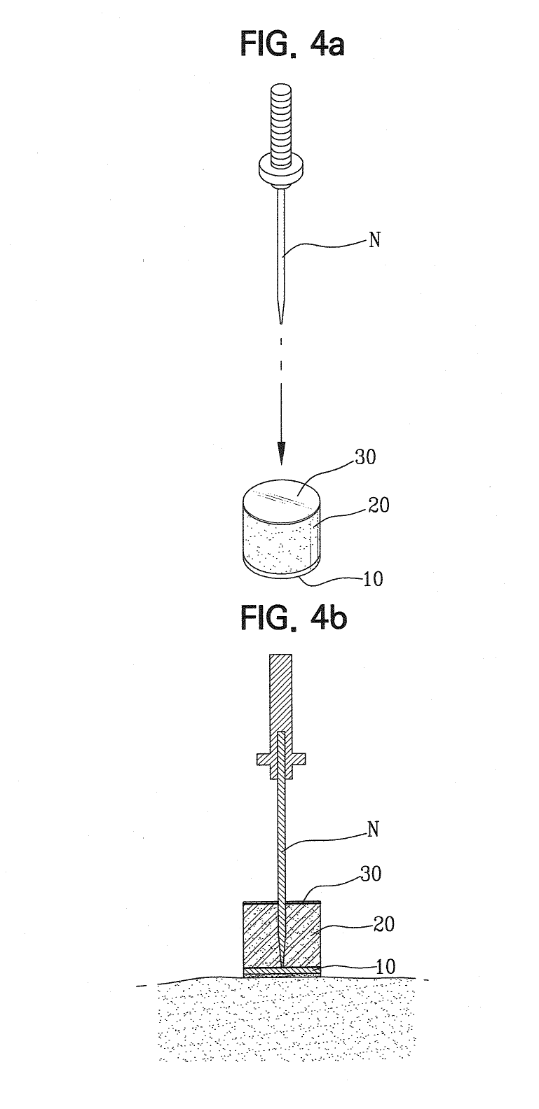

### DETAILED DESCRIPTION OF THE INVENTION

The pad for imaginary acupuncture therapy according to the present invention and the method for preparing the same will be hereinafter explained in detail with the reference to the accompanying drawings.

The present invention relates to a pad for imaginary acupuncture therapy and the method for preparing the same which provides a curing effect by acupuncture simulation without inserting an acupuncture pin into the skin. The pad for imaginary acupuncture therapy according to the present invention comprises a cushion layer (20) in an upper part, in which an acupuncture pin (N) is inserted into, a hard blocking plate (10) for blocking the penetration of an acupuncture pin (N) into the skin in a lower part. An adhesive is applied to the bottom of the hard blocking plate (10). A protecting film (30) is laminated on the surface of the cushion layer (20) to monitor the usage of the pad on the base from the presence of holes which are caused by the penetration of acupuncture pin (N). A coating paper (11) is attached to the adhesive applied to the bottom of the protecting plate (10) in a manner of easy separation.

The cushion layer (20) is made by expanded resin or sponge with a density suitable for acupuncture and the protection film (30) is preferably a paper or a vinyl which allows the trace of acupuncture penetration to be easily confirmed.

The basic pad for imaginary acupuncture therapy according to the present invention is manufactured by laminating in order of a coating paper (11) in a suitable size, a blocking plate (10) with the same size as the coating paper where an adhesive is applied to the bottom in a manner of easy separation, a cushion layer (20) with the depth for inserting acupuncture pin (N), and a protection film (30) in a shape of a thin membrane. From said basic pad for imaginary acupuncture therapy, various shapes of separated pads are made by cutting out the blocking plate (10), the cushion layer (20) and the protecting film (30) in an integrated state excluding the coating paper (11) from the basic pad by using cutters (not shown) having various pad shapes.

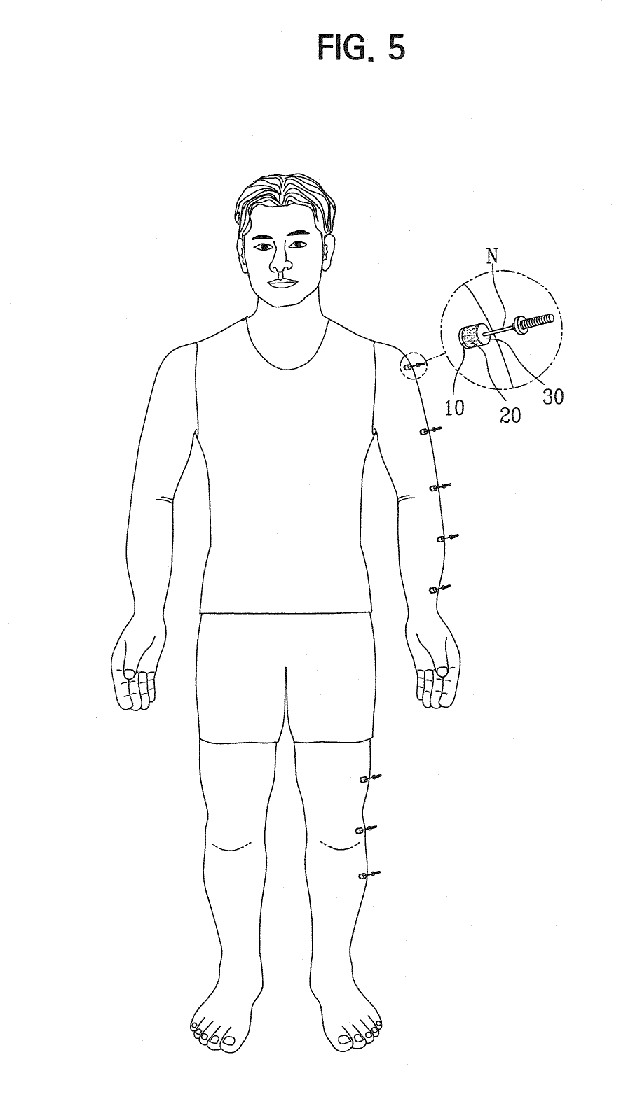

As shown in FIG. 5, several pads are taken out from the coating paper (11) to which they are attached, and the blocking plates (10) of the pads are attached to spots suitable for acupuncture distributed on the body. The acupuncture pin (N) is inserted through the protection film (30) exposed outside the cushion layer (20). Thus, imaginary acupuncture therapy by using the pad according to the present invention can cure a patient without accompanying any pain or fear.

The protecting film (30) of the pad for imaginary acupuncture therapy can confirm the reuse of the pad by checking a penetration hole caused by acupuncture and thus functions to prevent reuse of the pad in advance. Therefore, the pad for imaginary acupuncture therapy according to the present invention can prevent secondary infection and unsanitariness caused by reusing the pad with other patients by employing the protecting film (30). Also, in the pad for imaginary acupuncture therapy according to the present invention, as the cushion layer (20) is blocked by the protecting film (30), crumbled materials which may occur in the process of handling the pad due to damage to cushion layer (20) are not scattered. Thus, the pad for imaginary acupuncture therapy according to the present invention allows acupuncture therapy to be performed in a clean condition.

What is claimed is:

1. A pad for an imaginary acupuncture therapy comprising:
a cushion layer in an upper part, into which an acupuncture pin is inserted,
a blocking plate for blocking the acupuncture pin from penetrating into the skin in a lower part of the pad, and which is attached to a spot on a body suitable for acupuncture,
an adhesive applied to a bottom of the blocking plate, and
a protecting film on the cushion layer for confirming reuse of the pad.

2. A method for preparing a pad for an imaginary acupuncture therapy comprising the steps of:
preparing a basic pad by lamination in order of a coating paper in a suitable size, a blocking plate with the same size as the coating paper where an adhesive is applied to a bottom of the blocking plate, a cushion layer with a depth for inserting an acupuncture pin therein, and a protection film in a shape of a thin membrane, and
then cutting the blocking plate, the cushion layer and the protecting film in an integrated state, excluding the coating paper, from the basic pad by using cutters having various pad shapes.
